(19) United States
(12) Patent Application Publication
(10) Pub. No.: US 2026/0308104 A1
(43) Pub. Date: Oct. 8, 2026

(54) METHOD FOR PRODUCING METHYLAMINES IN THE GAS PHASE WITH IMPROVED CONTROL OF THE REACTOR INLET TEMPERATURE

(71) Applicant: BASF SE, Ludwigshafen am Rhein (DE)

(72) Inventors: Heiner SCHELLING, Ludwigshafen am Rhein (DE); Hans-Juergen PALLASCH, Ludwigshafen am Rhein (DE); Franz BORGEL, Ludwigshafen am Rhein (DE)

(21) Appl. No.: 19/477,831

(22) PCT Filed: Apr. 15, 2024
(86) PCT No.: PCT/EP2024/060120

§ 371 Date: Oct. 23, 2025

(30) Foreign Application Priority Data
Apr. 24, 2023 (EP) 23169421.7

(51) Int. Cl. C07C 209/60; C07C 209/84

(52) U.S. Cl. CPC C07C 209/60; C07C 209/84

(57) ABSTRACT

The invention relates to a process for producing methylamines by a gas phase reaction of methanol and ammonia as reactants, which is exothermic overall, in the presence of a heterogeneous catalyst at a pressure in the range from 15 to 35 bar. The reactants are evaporated in one or more heat exchangers and superheated before being fed to the reactor, where the heat exchangers are heated in turn with the hotter product gas stream from the reactor. In addition to methylamines (monomethylamine, dimethylamine and trimethylamine), the product gas stream also comprises unconverted reactants, water and any other reaction by-products. In order to control and limit the reactor inlet temperature to a temperature in the range from 350° C. to 410° C., a suitable quench liquid is introduced into the hot product gas stream before it is fed to the heat exchangers for the heating of the reactants. Evaporation and superheating of the added quench liquid removes the corresponding amount of energy is extracted from the hot product gas stream, as a result of which it is cooled efficiently and quickly. As a result, the temperature differential between the cold and hot sides of the heat exchangers is smaller, which means that less energy is transferred to the reactant stream and hence the reactor inlet temperature thereof can be adjusted or limited.

The present invention relates to a process for producing methylamines from methanol and ammonia in a continuous gas phase reaction over a heterogeneous catalyst at an absolute pressure in the range from 15 to 35 bar. For this purpose, the reactant stream, comprising the methanol and ammonia reactants, is evaporated by heat exchanger and heated to a reactor inlet temperature in the range from 350° C. to 410° C. Since the reaction of methanol and ammonia to give methylamines is exothermic, the hot product gas stream comprising not only the methylamines (monomethylamine, dimethylamine and trimethylamine) but also water, unconverted ammonia and unconverted methanol from the reactor is passed through one or more heat exchangers for heating of the reactants. It is a feature of the process of the invention that the reactor inlet temperature is adjusted by introducing a quench liquid into the hot product gas stream before it is passed through the heat exchangers. The quench liquid has a boiling point in the range from −40° C. to 120° C. at standard pressure and evaporates when introduced into the product gas stream, as a result of which it is cooled efficiently and in a controlled manner to the temperature required for the desired heating of the reactant stream. In a particular embodiment of the invention, condensate of the product gas stream is used as a quench liquid for the cooling of the product gas stream. The invention further relates to a corresponding apparatus for continuous performance of a reaction that is exothermic overall, for which the reactants have to be brought to a particular reactor inlet temperature, said apparatus comprising (a) one or more reactors to which the reactants are fed and from which the product gas stream is drawn off, (b) one or more heat exchangers upstream of the reactor(s), with which the reactants are brought to the required reactor inlet temperature and through which the product gas stream is passed for the introduction of heat, and (c) a measuring instrument for measurement of the reactor inlet temperature of the reactants. It is a feature of the apparatus of the invention that it comprises a controlled introduction of evaporable liquid into the product gas stream before it is passed through one or more of the heat exchangers, by means of which the temperature of the product gas stream is cooled by evaporation of the liquid and hence the desired reactor inlet temperature of the reactants can be established.

Methylamines are monomethylamine, dimethylamine and trimethylamine, and mixtures thereof.

Methylamines can be produced from methanol and ammonia in the presence of a catalyst in a continuous gas phase reaction. Although the reaction is exothermic overall, the reactants have to be preheated for the reaction. The reactants fed are typically preheated in heat exchangers upstream of the reactor. Preheating, as described in JP 60045550 A, can be accomplished using the product gas stream obtained in the reaction. For this purpose, the product gas stream is routed through the heat exchangers used for preheating. Because the reactions are exothermic overall, the temperature in the reactor increases between reactor inlet and reactor outlet. By virtue of the higher temperature at the reactor outlet, no further energy is required for preheating of the reactants when the product gas stream is used to heat the reactants in steady-state operation.

The required temperatures in the reactor are fixed by technical limitations. The reactor inlet temperature has to be sufficiently high for the conversion over the catalyst to set in immediately, for the methanol to be largely converted, and for the reaction to come close to thermal equilibrium. On the other hand, the temperature in the catalyst bed must not be too high since there will otherwise be increased breakdown of the methylamines to gaseous and solid degradation products, or the maximum permissible temperature of the reactor will be exceeded. Exact adjustment of the reactor inlet temperature is therefore crucial for efficient production of the methylamines.

The reaction of methanol and ammonia to give methylamines typically forms more trimethylamine than is required. Excess trimethylamine is typically recycled within the plant, added to the reactor feed and converted in the reactor in an endothermic reaction to dimethylamine and monomethylamine. The recycling of trimethylamine into the reactor thus leads to a less exothermic process overall. The same applies to the recycling of the other methylamines.

In the processes currently used for preheating of the reactants, temperature fluctuations in the environment or different amounts of recycled trimethylamine, and also changes in load, can result in fluctuations in the reactor inlet temperature. As a result, these fluctuations lead to fluctuations in the reactor temperatures. In the worst case scenario, the reactor temperature becomes so high that the methylamines break down, or the maximum permissible reactor temperature is exceeded.

The theoretical possibility of limiting the reactor inlet temperature of the reactant stream by diverting the product gas stream used for preheating around the heat exchangers when the upper temperature limit is attained is impractical for engineering reasons. To divert the large amount of hot and pressurized product gas stream quickly and in a controlled manner would require extremely complex and costly control valves.

WO 2005/028416 A describes a process in which controlled pressure regulation of the reactant streams to be heated or of the reactor output to be cooled is used to adjust the evaporation or condensation temperature such that exactly as much energy is transferred in the heat exchangers as required for attainment of the required reactor inlet temperature. Disadvantages of this process are that it is difficult to control, has slow closed-loop control characteristics and causes an additional pressure drop.

In addition, the process according to WO 2005/028416 A, in the case of elevated recovery of heat by preheating of the reactant streams with other suitable energy sources that are utilizable within the process, requires increased closed-loop control interventions and hence a further increase in pressure drop. In the case of part-load operation of the system, there may be an increase in specific heat transfer. In order to compensate for this increase in specific heat transfer, this process requires the performance of closed-loop control interventions that likewise cause an additional pressure drop.

It was therefore an object of the present invention to provide a process for producing methylamines from methanol and ammonia by gas phase reaction in which the reactor inlet temperature is adjusted quickly, efficiently and in a controlled manner within a narrow range and without a pressure drop. There should be a minimum amount of energy loss from the process through removal of heat, and a minimum amount of energy should have to be supplied externally for preheating and superheating of the reactants in steady-state operation.

Accordingly, the invention relates to a process for producing methylamines by continuous gas phase reaction, in which a reactant stream comprising the reactants methanol and ammonia is evaporated in one or more heat exchangers and optionally further heating elements and heated to a reactor inlet temperature in the range from 350° C. to 410° C., and is then fed to one or more reactors, where it is converted in the presence of a heterogeneous catalyst at an absolute pressure in the range from 15 to 35 bar to the methylamines monomethylamine, dimethylamine and trimethylamine, which are drawn off from the reactor as product gas stream together with water and any unconverted reactants, and with any by-products, wherein the reactor inlet temperature of the reactant stream is adjusted by operating at least one of the heat exchangers with the product gas stream which is cooled beforehand to the temperature required for the purpose by introducing a quench liquid having a boiling point in the range from −40° C. to 120° C. under standard pressure into the product gas stream.

The process of the invention for producing methylamine is thus based on the concept of lowering the temperature of the product gas stream coming from the reactor before it enters the heat exchanger to such an extent that the reactant stream is heated only to the desired reactor inlet temperature, with the required cooling of the product gas stream effected by introduction and evaporation of a suitable quench liquid, which evaporates completely. Liquids fundamentally suitable for this purpose are those that evaporate fully under the conditions of the product gas stream (i.e. at temperatures of at least 350° C. and an absolute pressure of 15 to 35 bar). This is the case for liquids having a boiling point in the range from −40° C. to 120° C. at standard pressure. This lowers the temperature of the product gas stream through the evaporation of the quench liquid without removal and loss of energy.

For production of methylamine from methanol and ammonia, the reactants are first evaporated and superheated in one or more heat exchangers. The reactants that have thus been heated to a temperature in the range from 350° C. to 410° C., preferably in the range from 350° C. to 390° C. and more preferably in the range from 360° C. to 380° C. are supplied to the reactor at that temperature (reactor inlet temperature).

In addition to methanol and ammonia, the reactor is typically supplied with the methylamines that forming in the reaction to an extent that exceeds the current demand. In particular, it may be necessary to recycle trimethylamine, which is typically formed in the reaction to a greater extent than required, to the reaction and hence increase the proportion of monomethylamine and dimethylamine in the methylamines for the overall process. Such recycling of trimethylamine and the associated endothermic transmethylation reduces the exothermicity of the overall reaction in the reactor.

Because the reaction is exothermic overall, the temperature increases from reactor entry to reactor exit. In order to ensure that the temperature in the reactor does not rise above 450° C., the setting of the reactor inlet temperature of the reactant stream has to be observed. In this case, a comparatively low reactor inlet temperature has to be set when the reaction is run with low recycling of methylamines, i.e. with high exothermicity (preferably reactor inlet temperature of 350° C. to 370° C.), or a comparatively high reactor inlet temperature has to be set when the reaction is run with high recycling of methylamines, i.e. with low exothermicity (preferably reactor inlet temperature of 370° C. to 410° C.). Excess heat of reaction may be removed if necessary by cooling the reactor. The reaction in the reactor is preferably run in such a way that the product gas stream on exiting the reactor is at a temperature in the range from 390° C. to 450° C.

The reactants are converted in the reactor to monomethylamine, dimethylamine and trimethylamine in the presence of a heterogeneous catalyst at an absolute pressure in the range from 15 to 35 bar. This pressure range is typically applicable not just to the reactor but also to the entirety of the feeding of the reactant stream through the heat exchangers and the discharging of the product gas stream via the heat exchangers, including any liquid separators. Accordingly, there is preferably also an absolute pressure in the range from 15 to 35 bar at the site of introduction of the quench liquid into the product gas stream.

A product gas stream is extracted from the reactor and comprises, in addition to the methylamines monomethylamine, dimethylamine and trimethylamine, also water produced as a byproduct in the reaction. The product gas stream typically also comprises unconverted methanol, unconverted ammonia, and other by-products such as carbon monoxide and carbon dioxide.

The reaction can be effected in one or more reactors. The reaction is preferably effected in one reactor. If two or more reactors are used, they may be connected in parallel or in series, or in combinations thereof.

The heterogeneous catalyst is typically an aluminum oxide doped with silicon dioxide, for example the N7066 catalyst from BASF. The heterogeneous catalyst is typically used in fixed bed form.

The product gas stream can then be separated into the individual reaction products. Separation is typically effected by distillation in two or more distillation columns. Methylamine formation usually forms more trimethylamine than desired. The excess of trimethylamine can then be fed back to the reaction in the reactant stream. The reaction of trimethylamine with ammonia to give monomethylamine and dimethylamine or with monomethylamine to give dimethylamine is endothermic. Corresponding endothermic reactions occur when the other methylamines are recycled. For the endothermic reaction, a portion of the heat of reaction released in the exothermic reaction of ammonia and methanol to give methylamines is consumed. For that reason, the reactor can also be operated adiabatically.

In the case of release of heat for heating of the reactant stream, the product gas stream in the heat exchangers is cooled and at least partly condensed. The condensate can preferably be separated from the gaseous components of the cooled product stream by liquid separator. This may be a separate liquid separator downstream of the heat exchangers or, preferably, liquid separators integrated into the heat exchangers. By means of such liquid separators (condensate separators) that are integrated or directly downstream of the individual heat exchangers, it is possible to avoid erosion by entrained condensate droplets. The liquid product streams obtained in the condensate separators are preferably supplied, just like the product gas stream leaving the heat exchangers, to a further treatment for separation and optionally separation of the methylamines.

The heat required to start up the reaction can be supplied by additional heating of the reactants with externally heated heat exchangers or electrical heating elements. Should it be the case that the product gas stream does not sufficiently heat the reactant stream in steady-state operation, the missing amount of heat can likewise be supplied with externally heated heat exchangers or electrical heating elements.

Heat exchangers used for preheating of the reactant stream may be any of the types of heat exchanger known to the skilled person. Suitable heat exchangers include, for example, shell and tube heat exchangers, spiral heat exchangers or plate heat exchangers. Preference is given to using shell and tube heat exchangers. Shell and tube heat exchangers that are particularly preferred for this purpose are modified floating head heat exchangers. The heat exchangers used can be operated in cocurrent, countercurrent or crosscurrent. In addition, any combination of cross-, counter- and cocurrent known to the skilled person is possible. For example, when several heat exchangers are used, at least one heat exchanger can be operated in cocurrent, while the remaining heat exchangers operate in countercurrent. The preferred mode of operation of the heat exchangers for heating of the reactants is countercurrent. The heat exchangers preferably have an integrated condensate separator, for the removal of condensate which forms when the product gas stream in the heat exchanger is cooled.

In order to control the reactor inlet temperature, measured values are typically recorded at various points in the plant for producing methylamines. For example, before the methanol is mixed into the ammonia-containing reactant stream, the flow of the ammonia-containing reactant stream and the flow of the methanol stream can be measured. In addition, when the methanol is mixed into the ammonia-containing reactant stream at different positions, the flow rate of the individual methanol substreams can be measured. When the product gas stream is separated into multiple substreams, the flow rate can be measured in each substream. A flow rate measurement can also be used to measure the amount of any additional steam needed to heat the reactants.

For specific closed-loop control of the reactor inlet temperature, it is necessary to measure the reactor inlet temperature of the reactant stream. In addition to the reactor inlet temperature, it is also possible to measure the temperature of the ammonia-containing reactant stream, the methanol, temperatures within the reactor and the temperature of the product gas stream at the reactor outlet.

The process of the invention preferably comprises an automatic closed-loop control system which controls the introduction of quench liquid into the product gas stream via a control valve depending on the measured reactor inlet temperature. In addition, it is possible to include other measurements in the closed-loop control system, such as the flow rate of the various process streams, reactor outlet temperature and the temperature of the product stream after introduction of the quench liquid, in order to optimize the control of the reactor inlet temperature.

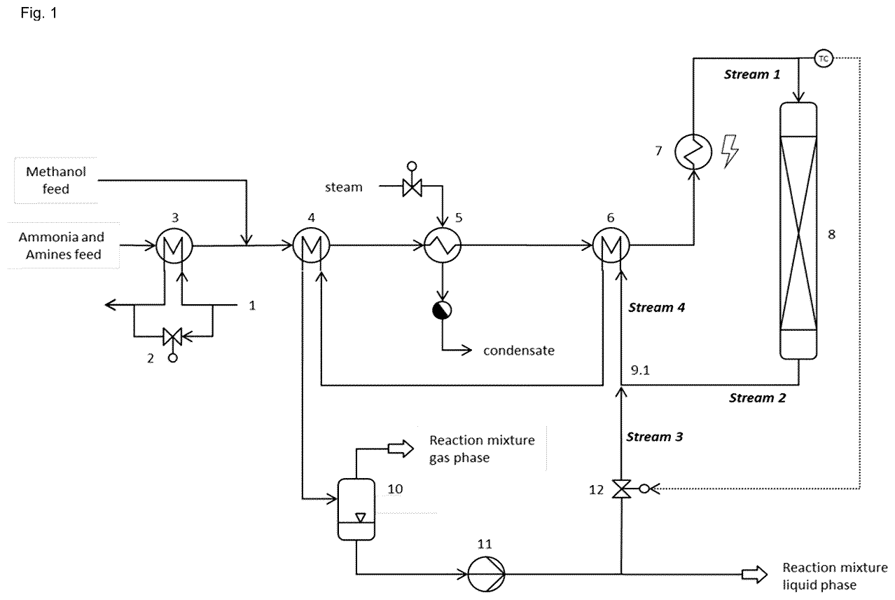

For an energy-efficient process for producing methylamines, it is advisable in steady-state operation both in partial load and full load to heat the reactant stream to reactor inlet temperature without the operation of externally heated heat exchangers or electrical heating elements, i.e. solely by means of heat exchangers that are heated by hot process streams, in particular the product gas stream. For example, it is possible in principle, in the first heat exchanger (3) of the process of FIG. 1, to heat and to partly evaporate the liquid mixture of ammonia and any recycled methylamines with an external stream, but preferably by means of a hot process stream to be cooled, for example, a hot wastewater stream from the process.

Owing to fluctuations in temperature in the environment, resulting from different amounts of recycled methylamines or other fluctuations in the operating regime, especially at partial load, and also fluctuations in the energy supplied to the preheaters (heat exchangers and electrical heating elements for heating, evaporation and superheating of the reactant stream), a correspondingly energy-efficient design of the process can lead to undesirably high reactor inlet temperatures. Efficient and rapid control of the reactor inlet temperature is therefore essential for an energy-efficient configuration of the process.

In the process of the invention for producing methylamines, the temperature of the product gas stream is cooled by the introduction and evaporation of a suitable liquid (quench liquid) to such an extent that the temperature of the reactants leaving the one or more heat exchangers heated by the product gas stream does not exceed the value required for intended operation of the reactor. A suitable quench liquid is a fluid having a boiling point in the range from −40 to 120° C. under standard pressure, preferably in the range from −35 to 105° C. under standard pressure.

Such a quench liquid may be a pure substance or else a mixture of two or more substances. Preferred quench liquids are the following substances that are already present in the process: ammonia, methanol, water, mono-, di- and trimethylamine and mixtures thereof. Particularly preferred quench liquids are substances or mixtures thereof which do not lead to any significant additional cost and inconvenience in the event of a subsequent separation of the reactor output, for example water, condensate generated in the cooling of the product stream, or bottoms draw streams from distillation columns in this subsequent separation. A very particularly preferred quench liquid is the condensate that arises in the heating of the reactant stream and the associated cooling of the product gas stream.

The quench liquid is preferably introduced into the product gas stream by injection by pump and control valve or by supply by control valve and motive jet nozzle or by hydrostatic supply via a control valve. Particular preference is given to injection by pump and control valve. In the case of injection by pump and control valve, the pump builds up the requisite positive pressure for the injection and the control valve controls the inlet into the product gas stream. In the case of hydrostatic supply, the elements of the system are arranged such that the quench liquid is fed into the product gas stream via the hydrostatic pressure. The control valve then controls the inlet into the product gas stream. When the quench liquid is introduced via a motive jet nozzle, the liquid is sucked in by the product gas stream itself and atomized in the nozzle. The inlet into the product gas stream is controlled here by a control valve. This variant is employable when the product gas stream has sufficiently high pressure.

The aim in respect of the quench liquid introduced is rapid evaporation. For this purpose, the quench liquid is preferably introduced into the product gas stream at a temperature that is only slightly below its boiling point at the operating pressure at the introduction site, more preferably at a temperature of not more than 20° C. below that boiling point and most preferably at a temperature of not more than 10° C. below that boiling point. In addition, for rapid evaporation, the quench liquid is preferably divided into small droplets with specifically high surface area by atomization in nozzles. Nozzles used may be nozzle types that are known for the purpose to the person skilled in the art, for example flat jet nozzles, hollow cone nozzles, full cone nozzles, spiral nozzles, fog nozzles or else motive jet nozzles. The nozzles may be combined here with a static mixer. Alternatively, the quench liquid can be distributed over large surfaces, such as random packings or structured packings as used in distillation technology. The flow through the random packings may be in co-, cross- or countercurrent to the flow direction of the quench liquid. In a preferred embodiment of the process, in which the quench liquid is introduced by injection by pump and control valve, the quench liquid to be introduced is distributed in the product gas stream by liquid distributor over a random packing or structured packing or preferably by means of a nozzle. In a preferred embodiment of the process, in which the quench liquid is introduced by hydrostatic supply, the quench liquid to be introduced is distributed in the product gas stream by liquid distributor over a random packing or structured packing.

Cooling of the product gas stream requires only small amounts of quench liquid.

The invention also provides a corresponding apparatus for continuous conversion of reactants to a product gas stream in a reaction that is exothermic overall, comprising
a) one or more reactors to which the reactants are fed and from which the product gas stream is drawn off,
b) one or more heat exchangers upstream of the reactors, with which the reactants are brought to the required reactor inlet temperature and through which the product gas stream is passed for the introduction of heat, and
c) one or more measuring instruments for measurement of the reactor inlet temperature of the reactants,
   wherein the apparatus comprises a controlled introduction for evaporable liquids into the product gas stream before it is passed through one or more of the heat exchangers. By means of this introduction, the temperature of the product gas stream can be cooled by evaporation of the liquid, heat exchange in the heat exchanger can be limited, and hence the desired reactor inlet temperature of the reactants can be established.

In a preferred embodiment, the apparatus of the invention comprises one or more liquid separators with which a condensate is separated from the product gas stream after or during passage through the heat exchangers, and a feed which supplies the condensate to the controlled introduction, where it is introduced into the product gas stream as evaporable liquid (quench liquid).

The invention accordingly provides an apparatus for continuous conversion of reactants to a product gas stream in a reaction that is exothermic overall, comprising
a) one or more reactors to which the reactants are fed and from which the product gas stream is drawn off,
b) one or more heat exchangers upstream of the reactors, with which the reactants are brought to the required reactor inlet temperature and through which the product gas stream is passed for the introduction of heat,
c) one or more measuring instruments for measurement of the reactor inlet temperature of the reactants, and
d) one or more liquid separators used to separate a condensate from the product gas stream after or during passage through the heat exchangers,
   wherein the apparatus comprises a controlled introduction of a portion of the condensate into the product gas stream before it is passed through one or more of the heat exchangers. By means of this introduction, the temperature of the product gas stream can be cooled by evaporation of the condensate, heat exchange in the heat exchanger can be limited, and hence the desired reactor inlet temperature of the reactants can be established.

In a preferred embodiment of the apparatus of the invention, the controlled introduction of the evaporable liquid or the condensate into the product gas stream comprises (a) a pump with control valve or (b) a control valve with motive jet nozzle or (c) a hydrostatic feed via a control valve. In order to assure faster evaporation of the evaporable liquid or the condensate and hence faster cooling of the product gas stream, the apparatus of the invention may comprise a nozzle for the introduction of the evaporable liquid or the condensate into the product gas stream. By means of the nozzle, the evaporable liquid or the condensate can be introduced into the product gas stream in the form of small droplets, which evaporate quickly because of their relatively large surface area. A nozzle is particularly suitable for introduction of condensate by pump and control valve. In addition, a nozzle is an inherent element of an introduction by control valve and motive jet nozzle. Alternatively, the apparatus of the invention, at the point at which the evaporable liquid or the condensate is introduced into the product gas stream, may also comprise a random packing or a structured packing over which the evaporable liquid or the condensate introduced is distributed by liquid distributor. The distributing of the evaporable liquid or the condensate over the large surface area of such a bed or packing likewise enables rapid evaporation. Such a bed or packing is especially suitable for introduction of evaporable liquid or condensate by hydrostatic feeding via a control valve, but also for introduction of evaporable liquid or condensate by pump and control valve. Accordingly, in this preferred embodiment,
in the case of introduction of the evaporable liquid or the condensate by pump with a control valve, the evaporable liquid or the condensate is distributed in the product gas stream via a liquid distributor and a random packing or structured packing or preferably via a nozzle, or
in the case of introduction of the evaporable liquid or the condensate by hydrostatic feeding via a control valve, the evaporable liquid or the condensate is distributed in the product gas stream via a liquid distributor and a random packing or a structured packing.

In a particular embodiment of the apparatus of the invention, the condensate passes through one or more purification units before being recycled into the product gas stream. These purification units are preferably distillation columns in which methylamines are withdrawn via the top, and the bottoms product therefrom is then returned to the product gas stream as condensate. Alternatively or additionally, condensate can also be introduced into the product gas stream which forms in such purification units from a product gas stream supplied in gaseous form, i.e., for example, the liquid bottoms product from a distillation column into which the gaseous product gas stream is fed and from which methylamines are drawn off via the top.

The invention is illustrated by the following drawings:

The drawings FIG. 1 to FIG. 3 show the flow diagrams of particular embodiments of the process of the invention for producing methylamines from methanol and ammonia.

### FIG. 1:

In the process for producing methylamines shown in FIG. 1, a first reactant stream (“Ammonia and Amines feed”) forms the feed to a first preheater (3). In addition to ammonia, the feed may also comprise recycled by-products and excess methylamines from the process. This reactant stream is heated in the first preheater (3). The heat supplied is provided by a hot process stream (1) to be cooled. In order to limit the transferred heat if necessary, the hot process stream can be diverted around the preheater (3) in full or in part by managing of the control valve (2). This preheated first feed is then mixed with the methanol feed (“Methanol feed”) and fed to the second preheater (4). This stream is also referred to hereinafter as reactant stream. The second preheater (4) is heated with the product gas stream (“Stream 4”). The reactant stream which is thus heated further is fed through a third preheater (5), which is heated with steam (“steam”). This serves mainly to heat up the reactant stream during the startup operation. It is also used when insufficient heat is transferred in the two upstream preheaters (3 and 4). In the fourth preheater (6), the reactant stream is superheated to the reactor inlet temperature. This preheater (6) is heated with the product gas stream (“Stream 4”) coming from the reactor. The reactant stream heated to reactor inlet temperature finally flows through a last electrically operated preheater (7) before being fed to the reactor (8) (“Stream 1”). This last preheater (7) serves mainly to heat up the reactant stream during the startup operation. It is also used when the reactant stream in the other preheaters does not reach the necessary reactor inlet temperature. Thus, while the first preheaters (3, 4, 5 & 6) are heat exchangers heated with external (5 & if necessary 3) or internal (4, 6 & if necessary 3) streams, the last preheater (7) and an electrical heating element. What takes place in the reactor (8) is not only the exothermic conversion of methanol and ammonia to monomethylamine, dimethylamine and trimethylamine but also the conversion of recycled trimethylamine which is not needed to dimethylamine and monomethylamine in an endothermic reaction. Overall, however, the reactions that proceed in the reactor (8) are exothermic. For that reason, the temperature increases across the reactor (8). The temperature of the reactor output (“Stream 2”) is higher than that required in the reactor feed (“Stream 1”). Therefore, the product gas stream of the reactor output in the preheaters (6 & 4) can be used to heat the reactor feed. For this purpose, the product gas stream (“Stream 4”) is directed in countercurrent through the fourth preheater (6) and the second preheater (4). Downstream of the second preheater (4), the cooled and now biphasic product stream is separated in a liquid separator (10) into a liquid phase and a gaseous phase. The gas stream leaving the liquid separator (10) is sent to workup. A portion of the condensate (“Stream 3”) from the liquid separator (10) is injected into the reactor output (“Stream 2”) via a pump (11) and a control valve (12) at a point (9.1) upstream of the fourth preheater (6). The injected liquid evaporates and removes the required heat of evaporation from the reactor output, thereby lowering the temperature of the product gas stream (“Stream 4”) leading to the fourth preheater (6). Because of the resultant reduction in temperature, a smaller amount of heat is transferred in the fourth preheater (6) to the reactor feed to be heated. Together with a temperature measurement in the feed to the reactor (8), it is thus possible to set up closed-loop temperature control. This ensures fast, immediate control of the reactor inlet temperature. The condensate from the liquid separator (10) which is not required for temperature control is likewise sent to the workup of the methylamines.

In addition to the execution variant described here with a total of five preheaters (3, 4, 5, 6 and 7), a multitude of variations in terms of number, arrangement and interconnection of heat exchangers for preheating the reactant stream are possible.

Instead of countercurrent operation of the preheaters as shown here, cocurrent or crosscurrent operation is also possible, as is any combination of countercurrent, cocurrent or crosscurrent.

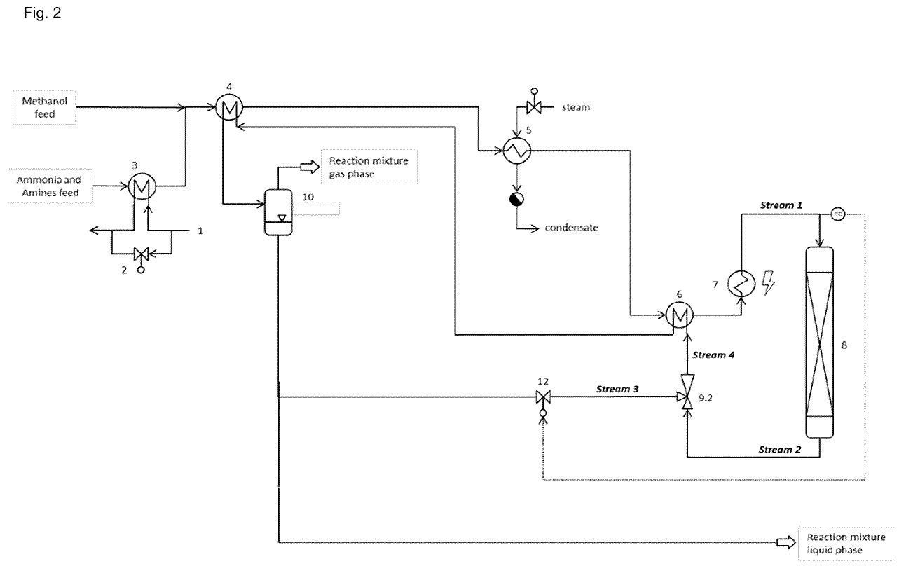

### FIG. 2:

The process shown in FIG. 2 is a variant of the process for producing methylamines as shown in FIG. 1, in which the liquid from the liquid separator (10) is sucked in via a motive jet nozzle (9.2) and atomized in the nozzle. The motive medium used here is the product gas stream (“Stream 2”) itself. This variant can be employed when the product gas stream has sufficiently high pressure.

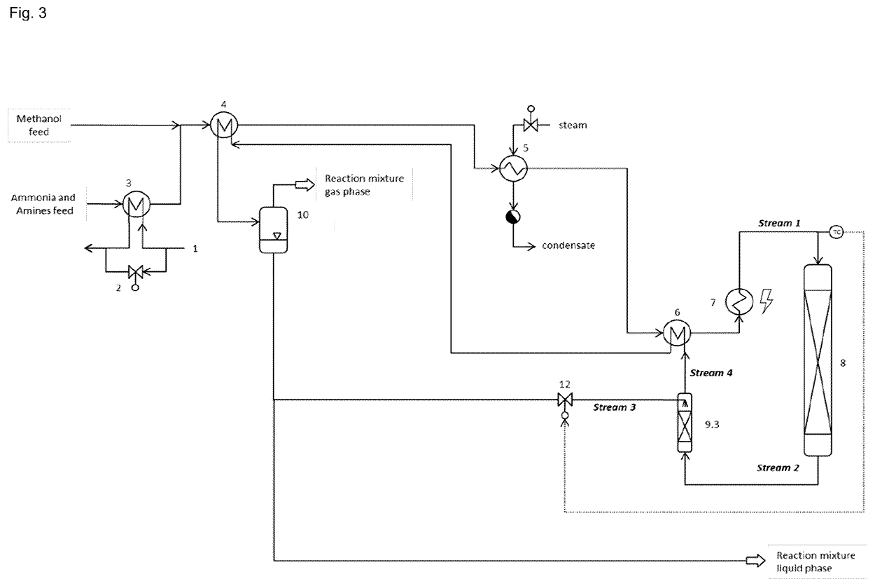

FIG. 3: The process shown in FIG. 3 is a variant of the process for producing methylamines as shown in FIG. 1, in which the preheaters are arranged in the system in such a way that the liquid from the liquid separator (10) can be introduced hydrostatically into the product gas stream (“Stream 2”). In order to create a maximum liquid surface area, the liquid is distributed by liquid distributor over a random packing or structured packing (9.3), as also correspondingly known from distillation technology.

### EXAMPLES

The example that follows illustrates the effect of the inventive introduction of quench liquid into the product gas stream of a process for producing methylamines as described in detail in FIG. 1 to FIG. 3. The reactor output (product gas stream) typically has a composition as shown in tab. 1.

This composition may vary depending on the design and mode of operation of the process.

TABLE 1
Typical composition of the product gas stream for the
process of the invention for producing methylamines

| | | |
|---|---|---|
| Hydrogen | <1.0% | by wt. |
| Nitrogen | <1.0% | by wt. |
| Ammonia | 25-37% | by wt. |
| Dimethyl ether | <1.0% | by wt. |
| Monomethylamine | 9-13% | by wt. |
| Dimethylamine | 11-16% | by wt. |
| Trimethylamine | 23-35% | by wt. |
| Methanol | <2.5% | by wt. |
| Water | 14-18% | by wt. |

A typical reactor inlet temperature is 360° C., for example. In the case of an adiabatic increase in temperature in the reactor of about 50° C. to 80° C., as is customary for the process, the result is a typical reactor outlet temperature of 410° C. to 440° C. Thus, a lowering of the reactor output (product gas stream) temperature of 10° C., for example, already leads to a significant reduction in the temperature differential between the feed and output stream and hence to a likewise considerable reduction in the energy transferred in the preheater zone. According to the invention, such a lowering of the temperature is achieved by introducing a suitable quench liquid into the product gas stream. Based on the process conditions (reactor output: 20.8 bar, 427.2° C., temperature of the quench liquid: see table 2) and the thermodynamic data of product gas stream and introduced quench liquid, the amount of quench liquid required to correspondingly cool down a particular amount of product gas stream can be determined. These results are compiled in tab. 2 for various possible quench liquids.

TABLE 2
Determination of the amount of liquid to be introduced into the reactor output for various
possible quench liquids that is required to reduce the temperature of the reactor output by 10 ºC.

| | | Amount |
|---|---|---|
| | Temperature | Quench fluid |
| Quench fluid | Quench fluid ° C | $\frac{kg}{( t reactor output * 10 ° C )}$ |
| Ammonia | 40-50 | 14.6-15.8 |
| Monomethylamine | 75-85 | 18.1-19.5 |
| Dimethylamine | 100-110 | 22.2-24.0 |
| Trimethylamine | 105-115 | 25.1-27.0 |
| Methanol | 155-165 | 18.9-20.5 |
| Water | 200-210 | 10.9-11.8 |
| Condensate | 83-93 | 14.2-15.9 |

Condensate here means the condensate obtained when the reactor output in the heat exchangers of the preheater section is cooled by heat transfer to the reactor feed. In the process as described in FIGS. 1 to 3, condensate is used to reduce the temperature of the product gas stream. In principle, however, the other quench liquids listed in tab. 2 are also useful for reducing the temperature.

For a selected state of operation of the process according to FIG. 1, tab. 3 collates, as an example, the data (compositions, pressure, temperature and state of matter) for cooling of the reactor output from 427.2° C. to 370° C. with condensate.

TABLE 3
Compositions and physical parameters of the streams (Stream 1
to 4) for performance of the process for producing methylamines
as shown in FIG. 1 in which the product gas stream is cooled
by introducing condensate to such an extent that the required
reactor inlet temperature of the reactant stream is assured.

| | | Stream | Stream | Stream | |
|---|---|---|---|---|---|
| | Molar | 1 | 2 | 3 | Stream |
| | mass | % by | % by | % by | 4 |
| | (g) | wt. | wt. | wt. | wt % |
| Hydrogen | 2 | 0.00 | 0.01 | 0.00 | 0.01 |
| Nitrogen | 28 | 0.01 | 0.05 | 0.00 | 0.05 |
| Ammonia | 17 | 38.92 | 30.18 | 24.36 | 29.71 |
| Dimethyl ether | 46 | 0.09 | 0.09 | 0.05 | 0.09 |
| Monomethylamine | 31 | 4.34 | 11.05 | 11.65 | 11.10 |
| Dimethylamine | 45 | 0.96 | 13.25 | 13.49 | 13.27 |
| Trimethylamine | 59 | 27.65 | 28.93 | 22.41 | 28.41 |
| Methanol | 32 | 27.00 | 0.54 | 0.92 | 0.57 |
| Water | 18 | 1.03 | 15.91 | 27.12 | 16.80 |
| Σ | kg/h | 1000.00 | 1000.00 | 85.92 | 1085.92 |
| Pressure (abs) | bar | 21.8 | 20.8 | 27.5 | 20.8 |
| Temperature | ° C. | 360.0 | 427.2 | 88.0 | 370.0 |
| State | | gaseous | gaseous | fluid | gaseous |

The lowering of the temperature as shown in this example reduces the temperature differential between the process stream leaving the last heat exchanger and the process stream entering the heat exchanger from 67.2° C. to 10° C.

The amount of 85.92 kg of condensate per tonne of reactor output that is required for cooling by 57.2° C. corresponds to about 15 kg of condensate per tonne of reactor output per 10° C. of cooling.

1.-16. (canceled)

17. A process for producing methylamines by continuous gas phase reaction, in which a reactant stream comprising the reactants methanol and ammonia is evaporated in one or more heat exchangers and optionally further heating elements and heated to a reactor inlet temperature in the range from 350° C. to 410° C., and is then fed to one or more reactors, where it is converted in the presence of a heterogeneous catalyst at an absolute pressure in the range from 15 to 35 bar to the methylamines monomethylamine, dimethylamine and trimethylamine, which are drawn off from the reactor as product gas stream together with water and any unconverted reactants, and with any by-products, wherein the reactor inlet temperature of the reactant stream is adjusted by operating at least one of the heat exchangers with the product gas stream which is cooled beforehand to the temperature required for the purpose by introducing a quench liquid having a boiling point in the range from −40° C. to 120° C. under standard pressure into the product gas stream.

18. The process according to claim 17, wherein the reactor inlet temperature is measured continuously.

19. The process according to claim 17, wherein the reaction in the reactor is run in such a way that the product gas stream on exiting the reactor is at a temperature in the range from 390° C. to 450° C.

20. The process according to claim 17, wherein the evaporation and adjustment of the temperature of the reactants is effected by means of two or more heat exchangers, and the quench liquid is introduced into the product gas stream upstream of the first heat exchanger for the product gas stream.

21. The process according to claim 17, wherein the quench liquid comprises one or more components selected from the group consisting of monomethylamine, dimethylamine, trimethylamine, ammonia, methanol, water.

22. The process according to claim 17, wherein the product gas stream is fully or partly condensed and then the methylamines formed are purified via one or more distillation columns and optionally separated into fractions of individual methylamines or methylamine compositions.

23. The process according to claim 22, wherein a trimethylamine-comprising or trimethylamine-enriched fraction is recycled to the reaction in the reactor.

24. The process according to claim 17, wherein the quench liquid used is a liquid stream that occurs within the process.

25. The process according to claim 17, wherein the product gas stream is cooled and fully or partly condensed, and the quench liquid used is the resulting condensate.

26. The process according to claim 22, wherein the quench liquid used is a bottoms draw stream from one or more of the distillation columns.

27. The process according to claim 17, wherein the quench liquid is introduced into the product gas stream by
a) injection by pump and control valve,
b) supply by control valve and motive jet nozzle, or
c) hydrostatic supply via a control valve.

28. The process according to claim 27, wherein the injected quench liquid is distributed in the product gas stream by liquid distributor over a random packing or structured packing or preferably by means of a nozzle, or the hydrostatically supplied quench liquid is distributed in the product gas stream by liquid distributor over a random packing or structured packing.

29. An apparatus for continuous conversion of reactants to a product gas stream in a reaction that is exothermic overall, comprising
a) one or more reactors to which the reactants are fed and from which the product gas stream is drawn off,
b) one or more heat exchangers upstream of the reactors, with which the reactants are brought to the required reactor inlet temperature and through which the product gas stream is passed for the introduction of heat, and
c) one or more measuring instruments for measurement of the reactor inlet temperature of the reactants,
wherein the apparatus comprises a controlled introduction for evaporable liquids into the product gas stream before it is passed through one or more of the heat exchangers, by means of which the temperature of the product gas stream can be cooled by evaporation of the liquid, heat exchange in the heat exchanger can be limited, and hence the desired reactor inlet temperature of the reactants can be established.

30. The apparatus according to claim 29, wherein the apparatus of the invention comprises one or more liquid separators with which a condensate is separated from the product gas stream after or during passage through the heat exchangers, and a feed which supplies the condensate to the controlled introduction, where it is introduced into the product gas stream as evaporable liquid.

31. The apparatus according to claim 29, wherein the apparatus for the introduction of the evaporable liquid into the product gas stream comprises
a) a pump with a control valve,
b) a control valve with a motive jet nozzle, or
c) a hydrostatic feed via a control valve.

32. The apparatus according to claim 31, wherein
in the case of introduction of the evaporable liquid by pump with a control valve, the evaporable liquid is distributed in the product gas stream via a liquid distributor and a random packing or structured packing or preferably via a nozzle, or
in the case of introduction of the evaporable liquid by hydrostatic feeding via a control valve, the evaporable liquid is distributed in the product gas stream via a liquid distributor and a random packing or a structured packing.
